(19) United States
(12) Patent Application Publication
(10) Pub. No.: US 2026/0248446 A1
(43) Pub. Date: Aug. 27, 2026

(54) Orthonan

(71) Applicant: Camilla Nicole Coleman, Birmingham, AL (US)

(72) Inventor: Camilla Nicole Coleman, Birmingham, AL (US)

(21) Appl. No.: 19/063,019

(22) Filed: Feb. 25, 2025

(51) Int. Cl. A61B 5/00; A61B 5/11; A61B 5/145; A61B 5/1468; A61B 90/00

(52) U.S. Cl. CPC A61B 5/45; A61B 5/11; A61B 5/14546; A61B 5/1468; A61B 5/746; A61B 5/7475; A61B 90/06; A61B 2560/045; A61B 2562/0219

(57) ABSTRACT

The present invention discloses a novel system for obtaining real-time images of bone and ligament movement by integrating an electrochemical biosensor, an accelerometer, and a piezoelectric transducer, utilized in conjunction with enzyme-based gold nanoparticles. The electrochemical biosensor detects specific biochemical markers related to musculoskeletal health, while the accelerometer measures dynamic motion and loads, and the piezoelectric transducer captures mechanical strain. The enzyme-based gold nanoparticles enhance the sensitivity and specificity of biochemical detection, providing valuable imaging contrast. This comprehensive system enables simultaneous monitoring of biochemical and biomechanical parameters, facilitating improved diagnostic capabilities in assessing musculoskeletal conditions. By delivering real-time insights into bone and ligament health, this invention offers significant advancements in personalized rehabilitation and treatment strategies, promoting optimal patient outcomes.

### BACKGROUND OF THE INVENTION

This invention pertains to a wireless wearable system designed for the real-time imaging and analysis of bone and ligament movement. The evaluation of musculoskeletal health has traditionally relied on invasive and time-consuming methods, such as imaging techniques (e.g., MRI, CT scans) or subjective assessments through physical examinations. These methods often do not provide real-time feedback regarding the biomechanics of bone and ligament movement, hindering timely intervention and rehabilitation. Moreover, existing monitoring systems rarely incorporate simultaneous biochemical and biomechanical analysis, limiting their ability to provide a comprehensive understanding of musculoskeletal conditions.

Currently, there is a growing demand for technologies that can deliver immediate, actionable insights into the biomechanics of bones and ligaments while simultaneously assessing the biochemical environment within these tissues. The integration of advanced sensing technologies, such as electrochemical biosensors and piezoelectric transducers, with novel materials like enzyme-based gold nanoparticles, presents a unique opportunity to meet this challenge.

The culmination of these advanced technologies into a single platform enables healthcare professionals and researchers to monitor musculoskeletal health more effectively, ultimately leading to improved treatment strategies, personalized rehabilitation protocols, and reductions in the reliance on invasive diagnostic techniques.

Thus, this invention represents a significant advancement in the field of musculoskeletal monitoring and management, combining real-time biomechanical assessment with biochemical analysis. The innovative integration of these technologies will provide a new paradigm for diagnosing and treating musculoskeletal disorders, enhancing patient outcomes, and revolutionizing approaches to rehabilitation.

### SUMMARY OF THE INVENTION

The present invention relates to a novel system designed for obtaining real-time images of bone and ligament movement through the integration of an electrochemical biosensor, an accelerometer, a piezoelectric transducer, and enzyme-based gold nanoparticles. This innovative approach enables simultaneous monitoring of both biochemical and biomechanical parameters, providing a comprehensive assessment of musculoskeletal health.

The electrochemical biosensor detects specific biochemical markers related to stress or injury within the tissues, releasing enzyme-based gold nanoparticles that enhance signal sensitivity and specificity. These nanoparticles facilitate biochemical imaging by interacting with the biological matrix, thus contributing valuable insights into tissue viability during movement.

In conjunction with the electrochemical biosensor, the accelerometer measures the dynamics of movements, capturing the loads and forces experienced by bones and ligaments. Simultaneously, the piezoelectric transducer detects mechanical strain, allowing for the visualization of real-time biomechanics.

By integrating these technologies, the invention enhances diagnostic capabilities for musculoskeletal conditions, enabling healthcare professionals to gather critical data on tissue health, optimize rehabilitation protocols, and improve patient outcomes without relying on invasive techniques. This system represents a significant advancement in the monitoring and management of musculoskeletal disorders, establishing a new paradigm for personalized patient care.

### DETAILED DESCRIPTION OF THE INVENTION

### System Components

### 1. **Wireless Wearable Electrochemical Biosensor**:

**Nanogap Nanowire Electrodes**: The biosensor incorporates nanogap nanowire electrodes, which enhance the detection sensitivity of biochemical markers relevant to bone and ligament health. This configuration allows for increased electrocatalytic activity, facilitating the monitoring of low concentrations of biomarkers and improving the overall specificity of measurements.
**Real-Time Data Transmission**: The electrochemical biosensor is equipped with wireless communication capabilities (e.g., Bluetooth or Wi-Fi) that enable the transmission of data to a mobile device or computer for immediate analysis and visualization.

### 2. **Accelerometer**:

The accelerometer is integrated into the wearable unit to continuously monitor the kinematics of bone and ligament movements. It captures data related to the acceleration, velocity, and direction of motion, which allows for a thorough understanding of the forces acting on these tissues during various activities.

### 3. **Piezoelectric Transducer**:

The piezoelectric transducer converts mechanical stress from bone and ligament movements into electrical signals, thereby enabling the real-time imaging of the biomechanics and motion of the tissue. This transducer is capable of detecting minute changes in strain and pressure, effectively illustrating how mechanical loads influence musculoskeletal dynamics.

### 4. **Painless Microneedle Patch**:

The system includes a painless microneedle patch designed to deliver enzyme-based gold nanoparticles transdermally. The microneedles are sufficiently small (typically ranging from 25 to 500 micrometers) to penetrate the uppermost layer of skin, allowing for the swift and comfortable release of nanoparticles into the tissue without causing pain or significant discomfort.
**Gold Nanoparticle Functionality**: The enzyme-based gold nanoparticles are engineered for specific biochemical interactions with markers associated with tissue health. These nanoparticles enhance the electrochemical signal detected by the biosensor and provide imaging contrast, crucial for visualizing biodynamic processes within the tissue.

### Functionality of the System

### 1. **Data Collection**

When the microneedle patch is applied, enzyme-based gold nanoparticles are released into the interstitial fluid surrounding the bone and ligament tissues. These particles bind to specific biochemical markers indicative of physiological conditions, triggering detectable electrochemical signals.
Simultaneously, the accelerometer records the motion data, while the piezoelectric transducer measures the mechanical strain experienced by the bone and ligament in real-time.

### 2. **Data Integration and Processing**

The signals acquired from the electrochemical biosensor, accelerometer, and piezoelectric transducer are transmitted wirelessly to a processing unit, which can either be a smartphone app or a dedicated computing device. Advanced algorithms are utilized to analyze these signals, converting raw data into meaningful insights regarding both biochemical and biomechanical conditions.
The piezoelectric transducer not only captures electrical signals but also generates real-time images based on the mechanical responses of the bone and ligament tissue, allowing for dynamic visualization of movement and strain.

### 3. **Visualization**

The combined information from the electrochemical biosensor and the mechanical data from the accelerometer and piezoelectric transducer is processed to create real-time visual representations of bone and ligament movements. The user interface presents graphical images displaying the biomechanical behavior and strain distribution in the tissues, allowing healthcare providers or researchers to assess tissue health and function immediately.

### Advantages of the Invention

**Real-Time Monitoring**: The system allows for continuous and immediate assessment of both biochemical and biomechanical parameters, enhancing clinical decision-making and rehabilitative strategies.
**Minimally Invasive**: The innovative microneedle patch delivery system minimizes discomfort, providing a non-invasive method for obtaining critical biochemical information.
**Enhanced Sensitivity and Resolution**: The use of nanogap nanowire electrodes substantially increases the electrochemical biosensor's sensitivity to biomarker detection, offering fine resolution in monitoring physiological changes.
**Integrated Biomechanics Imaging**: The piezoelectric transducer uniquely provides real-time images of biomechanics, illustrating how the tissues react under load and facilitating dynamic assessments of skeletal health.
**Comprehensive Data Presentation**: By combining multiple sensing modalities into one device, the invention offers a holistic approach to monitoring musculoskeletal conditions, significantly improving patient outcomes.

This invention provides a revolutionary approach to the assessment of bone and ligament health by integrating advanced sensing technologies into a single wireless wearable system. By leveraging the synergistic effects of nanotechnology and real-time monitoring, the system not only enhances diagnostic capabilities but also paves the way for personalized rehabilitation strategies and improved patient care in musculoskeletal health. The combination of biochemical and biomechanical analysis within a seamless, user-friendly platform represents a significant leap forward in wearable health technology.

1. A system for obtaining real-time images of bone and ligament movement, comprising:**
an electrochemical biosensor configured to detect specific biochemical markers associated with musculoskeletal health;
an accelerometer for measuring dynamic movements and loads experienced by said bone and ligament;
a piezoelectric transducer for detecting mechanical strain and generating electrical signals representative of biomechanical behavior;
enzyme-based gold nanoparticles that enhance signal sensitivity and specificity by interacting with said biochemical markers and providing imaging contrast.

2. The system of claim 1, wherein the electrochemical biosensor is capable of monitoring the release of the enzyme-based gold nanoparticles in response to detected biochemical markers.

3. The system of claim 1, wherein said enzyme-based gold nanoparticles are functionalized with ligands or antibodies specific to target analytes for enhanced detection of biochemical signals.

4. The system of claim 1, further comprising a processing unit configured to analyze data obtained from the electrochemical biosensor, accelerometer, and piezoelectric transducer to generate comprehensive real-time images of bone and ligament movement.

5. The system of claim 4, wherein the processing unit utilizes algorithms to integrate biochemical and biomechanical data for enhanced diagnostic capability regarding musculoskeletal conditions.

6. The system of claim 1, wherein the piezoelectric transducer is selected to provide high sensitivity to low mechanical strains experienced by said bone and ligament during movement.

7. The system of claim 1, wherein the accelerometer is configured to detect various movements, providing real-time load profiles of the bone and ligament during physical activities.

8. The system of claim 1, wherein the electrochemical biosensor and accelerometer are wirelessly connected to a user interface for real-time monitoring and visualization of collected data.

9. A method for assessing musculoskeletal health, comprising the steps of:
applying the system of claim 1 to a subject;
detecting biochemical markers using the electrochemical biosensor;
measuring dynamic movements with the accelerometer;
detecting mechanical strain with the piezoelectric transducer; and
integrating data from all components to produce real-time images reflecting the current state of bone and ligament movement.

10. The method of claim 9, wherein the use of enzyme-based gold nanoparticles facilitates enhanced imaging contrast and improves the sensitivity of the system to detect low concentrations of analytes.

11. A non-transitory computer-readable medium storing instructions that, when executed by a processing unit, cause the processing unit to perform the steps of claim 9.

12. The system of claim 1, wherein the electrochemical biosensor is designed to provide alerts when detected biochemical markers indicate potential stress or injury to the bone or ligament.
